(19) United States
(12) Patent Application Publication
(10) Pub. No.: US 2025/0134866 A1
(43) Pub. Date: May 1, 2025

(54) COMMENSAL SIGNALLING COMPOUNDS FOR TREATING OR PREVENTING MICROBIOME DYSFUNCTION IN THE SKIN OR MOUTH

(71) Applicants: Jan TRAMPOTA, Fairfield, NJ (US); Daniel B. YAROSH, Fairfield, NJ (US)

(72) Inventors: Jan TRAMPOTA, Fairfield, NJ (US); Daniel B. YAROSH, Fairfield, NJ (US)

(21) Appl. No.: 18/835,620

(22) PCT Filed: Feb. 2, 2023
(86) PCT No.: PCT/US2023/012237

§ 371 Date: Aug. 2, 2024

Related U.S. Application Data
(60) Provisional application No. 63/306,308, filed on Feb. 3, 2022.

(51) Int. Cl. A61K 31/4172; A61K 9/00; A61P 17/00

(52) U.S. Cl. CPC A61K 31/4172; A61K 9/0014; A61P 17/00

(57) ABSTRACT

Disclosed herein are methods for the treatment or prevention of topical or oral microbiome dysfunction in a subject in need thereof. The methods comprise the step of administering to the subject a compound capable of signaling the reduction of inflammatory response in the subject. In certain embodiments, the signaling compound is ergothioneine.

### CROSS-REFERENCE TO RELATED APPLICATION

This application claims priority to and the benefit of U.S. Provisional Application No. 63/306,308, filed on Feb. 3, 2022, the entirety of which is hereby incorporated by reference for all purposes.

### FIELD

The present disclosure sets forth methods for treating or preventing microbial dysfunction in a subject in need thereof. In particular, disclosed herein are methods of treating or preventing microbial dysfunction in the skin or oral cavity of a subject in need thereof by administering a commensal signaling compound to the subject in an amount effective to treat or prevent the dysfunction.

### BACKGROUND

The human microbiome is a collection of microbes, primarily bacteria and fungi, that reside on and in the human body. They fall into three broad categories. Commensals include microbes that are benign to human health. Pathogens include microbes that, if allowed to grow without control, can produce tissue damage and provoke an inflammatory immune response. Opportunistic pathogens include those that are benign under normal conditions but if they find a hospitable environment can grow, produce tissue damage and provoke an inflammatory immune response. The three main body sites where the microbiome resides are the gut, the mouth, and the skin.

In contrast to the gut and the mouth, the skin can provide a harsh external environment and relatively poor nutritional sources. There are three main types of skin niches for the microbiome: dry (such as on the forearms), moist (such as the axilla-armpits) and oily (such as the T-zone of the face-the forehead, nose and chin). Thousands of distinct species of microbes have been recovered from the skin. Many of these are difficult or impossible to culture in the laboratory, and most have not had their genomes sequenced.

Traditionally, the focus has been on the pathogens of the skin microbiome. Many products have been used that are antibiotic to kill skin microbes, or soaps and washes to remove them from the skin. Recently, a more sophisticated understanding of the microbiome has led to the recognition that dysbiosis, an imbalance in the relative populations of these microbiome species, is associated with unhealthy skin conditions and diseases. A working hypothesis is that changes in the diversity of the microbiome can lead to these conditions and diseases. For example, the skin disease atopic dermatitis has been characterized by microbial dysbiosis, with an reduction of microbial diversity and an overrepresentation of pathogenic Staphylococcus aureus (Koh et al, 2021, Allergology International 71(1):31-39; Li and Yosipovitch, 2020, American Journal of Clinical Dermatology (2020) 21 (Suppl 1):S44-S50). Overall, dysbiosis of the microbiome has been associated with rosacea, atopic dermatitis, eczema, psoriasis, acne, and other skin diseases (Pistore et al., 2021, J. Mol. Sci. 2021, 22, 9846).

The mouth contains a complex oral microbiome, whose dysbiosis is associated with oral diseases, such as dental caries, gingivitis, periodontitis, and oral cancer. The mouth microbiome may influence diseases throughout the digestive track and body, such as diabetes, pancreatic cancer, rheumatoid arthritis, colorectal and esophageal cancer, cystic fibrosis, cardiovascular disease and Alzheimer's disease (Willis and Gabaldon, 2020, Microorganisms 2020; 8:308). Dysbiosis of the oral cavity can cause symptoms and discomfort independent of infection such as dental caries and periodontitis (Wade, 2013, Pharmacol. Res. 69(1):137-143).

To date, there is no clear understanding of how dysbiosis leads to adverse skin or oral conditions or diseases. The simplest idea is that the commensals occupy sites that exclude pathogens, and if they are displaced pathogens grow out of control. For example, increases in pathogen food sources (for example increases in major lipid classes such as fatty acids, ceramides and cholesterol) are elevated in atopic dermatitis where S. aureus are overabundant (see Li and Yosipovitch, 2020). This theory does not require any communication between microbes or between human cells and microbes. A greater understanding of microbiome chemistry could identify compounds and compositions capable of treating or preventing symptoms and conditions or diseases caused by microbial dysbiosis.

### SUMMARY

These and other needs are addressed by the methods provided herein. The methods described herein are based, at least in part, on the discovery of signaling compounds and receptors or transporters produced by the microbiome of the skin and mouth. These signaling compounds are capable of reducing the inflammatory response in host cells and tissue, for instance in the skin and mouth. As demonstrated in the examples herein, administering one of the compounds is effective to treat or prevent inflammation in a model system.

In one aspect, provided herein are methods for treating or preventing microbiome dysfunction in the skin or mouth of a subject in need thereof. The methods comprise the steps of administering a nonhuman bacterial or fungal anabolite to the skin or mouth of the subject in an amount effective to treat or prevent the microbiome dysfunction. Useful anabolite compounds, compositions, methods of administration, and methods of occluding are described herein. In certain embodiments, the anabolite compound is ergothioneine, or a derivative thereof.

In certain embodiments, the methods are useful for treating or preventing rosacea, atopic dermatitis, eczema, psoriasis, sensitive skin, seborrheic dermatitis, dental caries, gingivitis, and periodontitis.

### DETAILED DESCRIPTION

Set forth herein are methods for treatment or prevention of microbiome dysfunction in a subject in need thereof.

### A. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures known in the art that are described or referenced herein are generally well understood and commonly employed using conventional methodologies by those skilled in the art.

As used herein, the singular forms “a,” “an,” and “the” include the plural referents unless the context clearly indicates otherwise.

As used herein, the term “about” refers to the stated value plus or minus 10%, plus or minus 5%, or plus or minus 1%. For example, a value of “about 10” can encompass a range of 9 to 11. For logarithmic scales, the term “about” refers to the stated value plus or minus 0.3 log units, or plus or minus 0.2 log units, or plus or minus 0.1 log units. For example, a value of “about pH 4.6” can encompass a pH range of 4.5-4.7.

“Ergothioneine” refers to ergothioneine in any form recognized by those of skill in the art, including derivatives of ergothioneine, and salts of ergothioneine and derivatives. In particular embodiments, ergothioneine, or a derivative thereof, is selected from the group consisting of L-ergothioneine, beta-hydroxyergothioneiene, gastrolathioneine, S-methyl ergothioneine, and selenoneine. Ergothioneine may be obtained or produced by any method or source known to those of skill in the art, including commercial sources.

“Salt” refers to any salt of a compound provided herein. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl) benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxy benzoyl) benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

In certain embodiments, the salt of a compound provided herein retains its biological properties and is not toxic or otherwise undesirable for pharmaceutical use.

The term “substantially free of” or “substantially in the absence of” with respect to a composition refers to a composition that includes at least about 85 or 90% by weight, in certain embodiments at least about 95%, 98%, 99% or 100% by weight, of a designated enantiomer or stereoisomer of a compound. For example, “substantially free of” or “substantially in the absence of” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated enantiomer or stereoisomer of a compound. In certain embodiments, in the methods and compounds provided herein, the compounds are substantially free of other enantiomers or stereoisomers.

Similarly, the term “isolated” with respect to a composition refers to a composition that includes at least 85, 90%, 95%, 98%, and 99% to 100% by weight, of a designated compound, enantiomer, or stereoisomer, the remainder comprising other chemical species, enantiomers, or stereoisomers. For example, “isolated” with respect to a composition can refer to a composition that includes about 85%, 86%, 87%, 88%, 89%, 90%, 91%, 92%, 93%, 94%, 95%, 96%, 97%, 98%, 99%, or 100% by weight of a designated compound, enantiomer, or stereoisomer, the remainder comprising other chemical species, enantiomers, or stereoisomers.

As used herein, EC₅₀ refers to a dosage, concentration or amount of a particular test compound that elicits a dose-dependent response at 50% of maximal expression of a particular response that is induced, provoked or potentiated by the particular test compound.

As used herein, the IC₅₀ refers to an amount, concentration or dosage of a particular test compound that achieves a 50% inhibition of a maximal response in an assay that measures such response.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is refractory or non-responsive to current treatments for hepatitis C infection. In another embodiment, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent which is known to be useful for, or has been or is currently being used for the treatment or prevention of a disorder or one or more symptoms thereof.

“Therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent or impede the onset, development, progression and/or severity of a disorder.

As used herein, the phrase “prophylactically effective amount” refers to the amount of a therapy (e.g., prophylactic agent) which is sufficient to result in the prevention or reduction of the development, recurrence or onset of one or more symptoms associated with a disorder (, or to enhance or improve the prophylactic effect(s) of another therapy (e.g., another prophylactic agent).

### B. Methods of Administration and Treatment or Prevention

The present disclosure provides methods for treating or preventing microbiome dysfunction in a subject in need thereof. The subject can be any subject experiencing or at risk for symptoms of microbiome dysfunction. In certain embodiments, the subject is a mammal. In certain embodiments, the subject is selected from a cow, pig, horse, cat, dog, rat, and mouse. In certain embodiments, the subject is a non-human primate. In certain embodiments, the subject is a human. In certain embodiments, the subject is an adult human. In certain embodiments, the subject is a pediatric human.

In the methods, the microbiome dysfunction can be in any cell or tissue of the subject deemed suitable by the practitioner of skill. In certain embodiments, the microbiome dysfunction is oral microbiome dysfunction. In certain embodiments, the microbiome dysfunction is skin microbiome dysfunction. The skin can be any skin of the subject deemed suitable by the practitioner of skill. In certain embodiments, the skin is dry. In certain embodiments, the skin is on the hands, feet, or limbs. In certain embodiments, the skin is moist. In certain embodiments, the skin is axillary skin. In certain embodiments, the skin is oily. In certain embodiments, the skin is facial skin. In certain embodiments, the skin is on the forehead, nose, or chin.

The administration can be for any purpose deemed suitable by the practitioner of skill. In certain embodiments, the methods are for therapy. In certain embodiments, the methods are for prophylaxis. In certain embodiments, the methods are for therapy and for prophylaxis.

In particular embodiments, the methods are for treating or preventing a disease or disorder in a subject in need thereof. In certain embodiments, the methods are for treating or preventing a disease or disorder selected from rosacea, atopic dermatitis, eczema, psoriasis, sensitive skin, seborrheic dermatitis, dental caries, gingivitis, and periodontitis.

In certain embodiments, the compound is an anabolite of a human microbiome organism. In certain embodiments, the compound is bacterial. In certain embodiments, the compound is fungal. In certain embodiments, the compound has little or no toxicity to the subject. In certain embodiments, the compound in an effective amount has no toxicity, or an acceptable amount of toxicity, to the subject, as deemed by a practitioner of skill. In certain embodiments, the compound is not naturally produced by the subject. The compound can be prepared or obtained by any technique deemed suitable. In certain embodiments, the compound is produced synthetically, for instance a synthetically produced compound that is otherwise naturally produced by an organism of the microbiome, for instance by a bacterium or by a fungus. In certain embodiments, the compound is sugar, amino acid, peptide, protein, vitamin, or polyphenol.

In certain embodiments, the compound has a receptor or transporter in the target cell or tissue of the subject, or a complex of proteins that function to transport the compound into the cell. In certain embodiments, the transporter is OCTN1. OCTN1 indicates the organic cation transporter, novel, type 1. It is also known as SLC22A4 (solute carrier family 22, member4) or ETT (the ergothioneine receptor). The human sequence can be found at NM_003059 (mRNA) and NP_003050 (protein). The mouse sequence can be found at NM_019687 or NM_001330304 (mRNA) and NP_062661 or NP_01317233 (protein). In certain embodiments, the receptor or transporter is expressed in skin cells of the subject. In certain embodiments, the receptor or transporter is expressed in oral cells of the subject, for instance epithelial cells.

In particular embodiments, the compound is ergothioneine, or a derivative thereof. Ergothioneine is 2S)-3-(2-Sulfanylidene-2,3-dihydro-1H-imidazol-4-yl)-2-(trimethylazaniumyl)propanoate, CAS registry no. 497-30-3. The ergothioneine can be ergothioneine in any form known to those of skill in the art. In particular embodiments, the ergothioneine is L-ergothioneine. In certain embodiments, the ergothioneine is a derivative of ergothioneine. The derivative can be any derivative of ergothioneine recognized by those of skill in the art. Particular derivatives include some chemical structure of ergothioneine and at least some function of ergothioneine. In particular embodiments, the derivative is selected from beta-hydroxyergothioneiene, gastrolathioneine, S-methyl ergothioneine, and selenoneine, and combinations thereof. Ergothioneine can be prepared or obtained by any method useful to those of skill in the art. In certain embodiments, ergothioneine is obtained commercially.

In certain embodiments, the compound is administered to the skin of the subject. The administration can be by any technique deemed suitable by the practitioner of skill. In certain embodiments, the administration is by direct application of a composition to the skin of the subject. In certain embodiments, the administration is with a wipe, swab, applicator, roll-on, stick, spray, or dropper. In certain embodiments, the compound is administered to the mouth of the subject. In certain embodiments, the administration is in a mouthwash or a mouth rinse.

The administration can be in any dose deemed suitable by the practitioner of skill. In certain embodiments, the dose is from about 0.1 mg to about 500 mg. In certain embodiments, the dose is from about 0.1 mg to about 100 mg. In certain embodiments, the dose is from about 0.1 mg to about 50 mg. In certain embodiments, the dose is from about 0.1 mg to about 40 mg. In certain embodiments, the dose is from about 0.1 mg to about 30 mg.

In certain embodiments, the administration is via a single application. In certain embodiments, the administration is via two applications. In certain embodiments, the administration is via three applications. In certain embodiments, the administration is via four applications. In certain embodiments, the administration is via three applications. In certain embodiments, the administration is via five applications. In certain embodiments, the administration is via six applications. In certain embodiments, the administration is via seven applications. In certain embodiments, the administration is via eight applications. In certain embodiments, the administration is via nine applications. In certain embodiments, the administration is via ten applications. In certain embodiments, the administration is via more than ten applications.

The administration can be for any length of time deemed suitable by the practitioner of skill. In certain embodiments, the compound is administered for 1-10 minutes, 1-3 minutes, 1-5 minutes, or for about 1 minute. In certain embodiments, the administration is via a wipe, roll-on, stick, or other applicator.

The administration can be repeated as deemed needed by the practitioner of skill. In certain embodiments, the administration is once per day. In certain embodiments, the administration is twice per day. In certain embodiments, the administration is three times per day. In certain embodiments, the administration is four times per day. In certain embodiments, the administration is 2-3 times per day for a few days followed by once per day for a few days. The number of days can be determined by the practitioner of skill.

The administration can continue for any length of time deemed suitable by the practitioner of skill. In certain embodiments, the administration is for one day. In certain embodiments, the administration is for one week. In certain embodiments, the administration is for two weeks. In certain embodiments, the administration is for three weeks. In certain embodiments, the administration is for four weeks. In certain embodiments, the administration continues as needed while symptoms are evident. The administration can be at any interval deemed suitable by the practitioner of skill. In certain embodiments, the administration is every day. In certain embodiments, the administration is every other day. In certain embodiments, the administration is four days per week. In certain embodiments, the administration is three days per week. In certain embodiments, the administration is two days per week. In certain embodiments, the administration is one day per week.

### Pharmaceutical and Nutraceutical Compositions and Routes of Administration

The compositions provided herein can be formulated as pharmaceutical compositions using methods available in the art and those disclosed herein. Any of the compositions disclosed herein can be provided in the appropriate pharmaceutical composition and be administered by a suitable route of administration. The compositions provided herein can also be formulated as nutraceutical or nutritional formulations with additives such as nutraceutically or nutritionally acceptable excipients, nutraceutically or nutritionally acceptable carriers, and nutraceutically or nutritionally acceptable vehicles.

The methods provided herein encompass administering pharmaceutical or nutraceutical compositions containing at least one compound as described herein, either used alone or in the form of a combination with one or more compatible and pharmaceutically acceptable carriers, such as diluents or adjuvants.

In clinical practice the compositions provided herein may be administered by any conventional route, in particular orally, parenterally, rectally or by inhalation (e.g. in the form of aerosols). In certain embodiments, the compositions provided herein are administered orally.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease.

In pharmaceutical compositions, use may be made, as solid compositions for oral administration, of tablets, pills, hard gelatin capsules, powders or granules. In these compositions, the active product is mixed with one or more inert diluents or adjuvants, such as sucrose, lactose or starch.

These compositions can comprise substances other than diluents, for example a lubricant, such as magnesium stearate, or a coating intended for controlled release.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmacopeia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, NY, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The composition, shape, and type of dosage forms provided herein will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing, Easton PA (2000).

Generally, the ingredients of compositions are supplied either separately or mixed together in unit dosage form, for example, as a dry lyophilized powder or water free concentrate in a hermetically sealed container such as an ampoule or sachet indicating the quantity of active agent.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound(s).

### Topical and Mucosal Dosage Forms

Also provided are topical and mucosal dosage forms. Compounds described herein may be formulated in products for application to the skin, hair, eyebrows and eyelashes, nails, lips, teeth, and gums. They may be formulated into moisturizers, serums, tonics, gels, creams, sprays, skin balms, lip balms, mouthwashes, teeth strips, sublingual pads, and such. They may be formulated into food or drinks.

Topical and mucosal dosage forms include, but are not limited to, solutions, sprays, aerosols, creams, lotions, ointments, gels, solutions, emulsions, suspensions, or other forms known to one of skill in the art. See, e.g., Remington's Pharmaceutical Sciences, 16^{th}, 18th and 20^{th} eds., Mack Publishing, Easton PA (1980, 1990 & 2000); and Introduction to Pharmaceutical Dosage Forms, 4th ed., Lea & Febiger, Philadelphia (1985). In certain embodiments, the dosage form is selected from creams, lotions, gels, ointments, serum, face and body powder, foundation, color makeup, eyeliner, mascara, antiperspirant, deodorant, and micro-sponge.

The products may be for treatment of dysbiosis of the microbiome resulting from natural variations in the skin condition, genetic predispositions to dysbiosis, unusual washing or removal of the microbiome from the skin, infections by pathogens, or misuse of probiotics. The products may be designed to reduce inflammation of the skin, scalp or gums, even skin tone, hydrate or moisturize the skin, reduce fine lines and wrinkles, improve skin and gum radiance and such.

Dosage forms suitable for treating mucosal tissues within the oral cavity can be formulated as mouthwashes or as oral gels. In certain embodiments, the dosage form is pharmaceutical. In certain embodiments, the dosage form is cosmetic.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th}, 18th and 20^{th} eds., Mack Publishing, Easton PA (1980, 1990 & 2000).

Depending on the specific tissue to be treated, additional components may be used prior to, in conjunction with, or subsequent to treatment with active ingredients provided. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### Dosage and Unit Dosage Forms

In human therapeutics, the doctor will determine the posology which he considers most appropriate according to a preventive or curative treatment and according to the age, weight, stage of the infection and other factors specific to the subject to be treated. In certain embodiments, doses for each compound are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of each active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a subject is 0.140 mg/kg to 3 mg/kg of the subject's body weight, based on weight of the active compound. In certain embodiments, the dosage administered to a subject is between 0.20 mg/kg and 2.00 mg/kg, or between 0.30 mg/kg and 1.50 mg/kg of the subject's body weight.

In certain embodiments, the recommended daily dose range of a composition provided herein for the conditions described provide each compound within the range of from about 0.1 mg to about 1000 mg per day, given as a single once-a-day dose or as divided doses throughout a day. In certain embodiments, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiment, the dosage of each compound of a composition provided herein, based on weight of the active compound, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising a compound, or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

In certain embodiments, the compound is administered with a second agent. The second agent can be any second agent deemed suitable to the person of skill. In certain embodiments, the second agent is a prebiotic, probiotic, or postbiotic. In certain embodiments, the second agent is selected from lactobacillus, bifidus cells and extracts, lactic acid, sodium lactate, citric acid, and sodium citrate. In certain embodiments, the second agent is an antioxidant. In certain embodiments, the second agent is selected form vitamin C, acrobyl glucoside, magnesium ascorbyl phosphate, CoQ10, idebenone, green tea, ECGC, vitamin E, tocopherol, tocopheryl acetate, and dipotassium glycyrrhizate. In certain embodiments, the second agent is an anti-inflammatory. In certain embodiments, the second agent is selected from bisabolol, indomethacin, ibuprofen, aloe, and caffeine. In certain embodiments, the second agent is an anti-aging agent. In certain embodiments, the second agent is selected from niacinamide, glycolic acid, alpha and beta hydroxy acids, retinol, retinaldehyde, retinal palmitate, ceramides, peptides, epidermal growth factor, stem cells, squalene, squalene, collagen, glycerin, hyaluronic acid, algae extract, yeast extract, molasses extract, and resveratrol. In certain embodiments, the second agent is an anti-acne agent. In certain embodiments, the second agent is selected from salicylic acid, hydrogen peroxide, and benzoyl peroxide. In certain embodiments, the second agent is an anti-pigmentation agent. In certain embodiments, the second agent is selected from retinol, vitamin C, arbutin, and hydroquinone. In certain embodiments, the second agent is a micronutrient. In certain embodiments, the second agent is selected from vitamin C, vitamin D, vitamin E, and vitamin K. In certain embodiments, the second agent is a preservative. In certain embodiments, the second agent is selected from phenoxyethanol, disodium EDTA, BHT, postassium sorbate, and sodium benzoate. In certain embodiments, the second agent is a mouthwash agent. In certain embodiments, the second agent is selected from fluoride, cetylpyridinium chloride, chlorhexidine, essential oils, carbamide peroxide, and hydrogen peroxide. In certain embodiments, there are multiple second agents, for instance combinations of the second agents described herein.

In certain embodiments, each active agent is administered separately. In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week part, at about 1 to 2 weeks apart, or more than 2 weeks apart.

In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, the compounds are administered to a patient, for example, a mammal, such as a human, in a sequence and within a time interval such that the compound provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In certain embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In certain embodiments, the compounds provided herein are cyclically administered to a patient. Cycling therapy involves the administration of a first agent for a period of time, followed by the administration of a second agent for a period of time and repeating this sequential administration. Cycling therapy can reduce the development of resistance to one or more of the therapies, avoid or reduce the side effects of one of the therapies, and/or improve the efficacy of the treatment.

In certain embodiments, the compounds provided herein are administered in a cycle of less than about 3 weeks, about once every two weeks, about once every 10 days or about once every week. One cycle can comprise the administration of a compounds provided herein by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of each agent are administered separately yet within a time interval such that the compounds provided herein can work together. For example, one component can be administered once per week in combination with the other components that can be administered once every two weeks or once every three weeks. In other words, the dosing regimens are carried out concurrently even if the therapeutics are not administered simultaneously or during the same day.

### Kits

Also provided are kits for use in methods. The kits can include a compound or composition provided herein, a second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Assay Methods

Compounds can be assayed for amounts or activity according to any assay known to those of skill in the art.

### EXAMPLES

Unless otherwise stated, chemical reagents were purchased from commercially available sources.

Reagents used herein are available from commercial vendors and were purchased from commercially available sources unless specified herein otherwise or unless the preparation of the reagent(s) is/are described herein.

### Example 1

The present example provides the discovery of skin and oral microbiome organisms that produce ergothioneine (EGT). EGT is not made by human cells but is synthesized by some bacteria and fungi. To date the consensus of experts on the source of EGT in humans is the human diet, wherein bacteria and fungi produce EGT, which is taken up by plants and then animals and then consumed by humans, where it is absorbed through the gut.

The bacteria and fungi that synthesize EGT might be inferred from the presence in their genome of genes that code for specific EGT synthesizing enzymes, such as the egtA,B,C,D,E or the egt-1,2 genes. The consensus of experts in the field has been that EGT is not synthesized within the human gut, and no one has discussed synthesis of EGT on the skin. According to Cheah and Halliwell, 2021, Redox Biology 42:101868, “Deeper in silico analysis of bacteria from the phyla Bacteroidetes, Actinobacteria, Cyanobacteria and Proteobacteria, present in normal gut microflora, reveals that many of these bacteria appear to possess the egtBD genes, or homologs of them. This therefore raises the question as to whether gut microflora can produce ET [ergothioneine]. Although evidence suggests that this does not occur (reviewed in Melville, 1959, Vitam. Horm. 17:155), this likely needs re-examination using more sensitive and accurate methods for ET analysis.”

The discovery began with the recognition that, while some organisms have the complete 5-gene EGT synthetic cluster, only the egtB and egtD) genes are required to produce EGT, with other synthetic enzymes filling in the missing anabolic steps. This pair of EGT-specific genes are found in a number of diverse phyla (reviewed in Jones 2014). However, experts in the skin microbiome are discouraged by the relatively low proportion of skin microbes that have been identified and by the relatively low proportion of those organisms whose genomes have been sequenced. The difficulty of the analysis is compounded by the fact that there are no systematic lists of skin microbes and no systematic lists of species with egt-related genes by which to cross-check.

BLAST (basic local alignment search tool) analysis using the U.S. National Library of Medicine database of thousands of sequenced genomes identified 105 microbe species that have an exact match for the egtD gene and 211 species that have sequences closely related to the egtD gene. A few of the phyla of these organisms have been previously identified as containing EGT-specific genes, but not identified as skin or oral microbe species. This list was then compared to various published lists of skin and oral microbiome species. The following table shows skin and oral microbes grouped by phyla, with references (some contradictory) or BLAST discoveries.

TABLE 1
Microbes found in skin microbiome containing
at least one EGT-specific synthetic gene.

| Phyla/Genus & Species | Reference |
|---|---|
| Actinobacteria | egtA, B, D, E (Cumming et al., 2018, |
| | Antiox. Redox Signal. 28: 431) |
| Corynebacterium | No EGT (Cumming) |
| Corynebacterium halotolerans | egtB & D (Jones) |
| Propionibacterium | No EGT (Cumming); missing egtE (Jones |
| | et al, 2014, Gene 549: 161, 2014) |
| Actinomycetes | egtA, B, C, D, E (Cumming) |
| Streptomyces griseus | BLAST |
| Frankia casuarinae | BLAST |
| Mycobacteria avium | BLAST |
| Salinispora tropica | BLAST |
| Firmicutes | |
| Exiguobacterium sibiricum | BLAST |
| Proteobacteria | egtB, D (Cumming) |
| Paracocciodioides brasiliensis | egt1 (Jones) |
| Pseudomonas aeruginosa | BLAST |
| Xanthomonas oryzae | BLAST |
| Teredinibacter turnerae | BLAST |
| Paraburkholderia xenovorans | BLAST |
| Acidovorax avenae | BLAST |
| Caulobacter segnis | BLAST |
| Cupriavidus metallidurans | BLAST |
| Bacterioidetes | Some members have egtB, D (Jones) |
| Chryseobacterium | BLAST |
| vrystaatense | |
| Archae | |
| Thaumarcheota phylum | egtB, D (Jones) |
| Cenarchaeum symbiosum | egtB, D (Jones) |
| Candidatus Nitrosopumilus | egtB, D (Jones) |
| koreensis | |
| Candidatus Nitrosopumilus | egtB, D (Jones) |
| Nitrosopumilus maritimus | egtB, D (Jones) |

The oral microbiome is much less characterized, but bacteria from the phyla with egt genes, such as Actinobacteria, Bacterioidetes, Firmicutes as well as Archea, have been identified in the mouth (Wade, 2013, Pharmacol. Res. 69:137).

As demonstrated in the present example, the recognition that EGT-synthesis genes are widespread among skin and oral microbiome commensals is necessary but not sufficient for the invention.

### Example 2

The present example evaluates the effect of EGT on two bacterial strains.

The growth of two bacterial strains, one commensal containing EGT-synthetic genes (P. aeruginosa ATCC 27853) and one pathogen lacking such genes (S. Aureus ATCC 6538) were compared in the presence and absence of EGT. Each strain was adjusted to an OD₆₀₀ of 0.2 in MH culture media, inoculated into 96-well plates and incubated at 37 C. The growth of the cultures were monitored for optical density for 24 hours. The S. aureus and P. aeruginosa cultures reached stationary phase after 15 and 2 hours, respectively. Addition of L-ergothioneine (99+% purity) at 0.001%, 0.05% or 0.1% (w/v) upon inoculation had no effect on the growth rate of either culture.

This demonstrates that EGT is not acting as a nutrient or growth stimulant for these microbes, regardless of their EGT-gene status.

### Example 3

The present example examines the release of the inflammatory cytokine IL-8 by human keratinocytes after treatment with S. aureus. Although S. aureus is associated with atopic dermatitis, IL-8 is not identified as inflammatory cytokine in this disease (Li and Yosipovitch, 2020).

Infection of a keratinocyte culture at a multiplicity of infection of 10 increased the amount of IL-8 from low levels in uninfected cultures to an increase of 6,044% in 24 hours in infected cultures. Pre-treatment of the keratinocyte cultures with EGT for 24 hours reduced the release of IL-8 by 61% for 0.005% EGT, 75% for 0.01% EGT, and 82% for 0.05% EGT.

Of particular note here is that EGT was effective at very low concentrations (0.005%=50 parts per million), and concentrations beyond this showed little dose responsiveness. Both of these are characteristics of signaling molecules rather than antioxidant molecules consumed in free radical reactions.

### Example 4

The present example tests the effect of EGT on skin inflammation in subjects with atopic dermatitis lesions.

Three topical formulas were prepared using a commercial formulation of EGT called MC-2, which is a granulated preparation of EGT mixed into mannitol. One formulation was a placebo, with only mannitol added. The second was a low dose formula containing EGT in the form of MC-2 added to a final concentration of 0.005% EGT (w/w). The third was a high dose formulation containing EGT in the form of MC-2 added to a final concentration of 0.05% EGT (w/w).

Subjects with patches of atopic dermatitis were recruited to the study and 16 were assigned to each of three arms: placebo, low dose EGT and high dose EGT. The subjects were 9 males and 39 females, representing each Fitzpatrick Skin Type (I-VI) in age range from 21 to 73 years old. The redness of the skin lesion was measured using a dermospectrophotometer and compared to adjacent uninvolved skin to determine a redness measure. The subjects used the test formula on the skin lesion twice a day for 4 weeks. The lesions were again measured with a dermospectrophotometer.

At the start of the study the average redness of the skin lesion above the adjacent non-involved skin was 5.0 units for all subjects. After 4 weeks, the placebo group had a redness reduction of only 9%, while the low dose EGT group had a reduction of 66% and the high dose EGT group had a reduction of 50%.

Once again, of special note is that EGT was effective at very low concentrations of 50 parts per million, and increasing the concentration by 10-fold to 0.05% had no improvement on the reduction of redness. These results further support the discovery that EGT is acting as a signaling molecule rather than as an antioxidant.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A method of treating or preventing microbiome dysfunction in the skin or mouth of a human patient in need thereof comprising the step of administering to the skin of the human a nonhuman bacterial or fungal anabolite in an amount effective to treat or prevent the microbiome dysfunction.

2. The method of claim 1 wherein the anabolite has a human receptor or a human transporter.

3. The method of claim 2 wherein the human receptor or human transporter is found in human skin or human epithelial cells.

4. The method of any of the previous claims wherein the anabolite has little or no toxicity to the human patient.

5. The method of any of the previous claims wherein the anabolite is produced by a human skin bacterium.

6. The method of any of the previous claims wherein the anabolite is produced by a skin fungus.

7. The method of any of the previous claims wherein the anabolite is ergothioneine, or a derivative thereof.

8. The method of any of the previous claims wherein the anabolite is a sugar, amino acid, peptide, protein, vitamin, or polyphenol.

9. The method of any of the previous claims wherein the anabolite is ergothioneine.

10. The method of any of the previous claims wherein the anabolite is administered in the form of a composition comprising the anabolite and one or more carriers, excipients, or diluents.

11. The method of claim 10 wherein the composition is formulated as a topical composition.

12. The method of claim 10 wherein the composition is formulated as a lotion, cream, or ointment.

13. The method of claim 10 wherein the composition is formulated as an oral composition.

14. The method of claim 10 wherein the composition is formulated as a mouthwash.

15. The method of any of the previous claims wherein the dose of the anabolite is from 0.1 to about 500 mg, from about 0.1 to about 100 mg, from 0.1 to about 50 mg, from 0.1 to about 40 mg, or from 0.1 to about 30 mg.

16. The method of any of the previous claims wherein the microbiome dysfunction is a disease or disorder.

17. The method of claim 13 wherein the disease or disorder is selected from rosacea, atopic dermatitis, eczema, psoriasis, sensitive skin, seborrheic dermatitis, dental caries, gingivitis, and periodontitis.
